(19) United States
(12) Patent Application Publication
(10) Pub. No.: US 2012/0041477 A1
(43) Pub. Date: Feb. 16, 2012

(54) Device for Enhancing the Appearance of a Person

(76) Inventor: Kitty Nancy Van Rijswijk, Cape Town (ZA)

(21) Appl. No.: 13/148,997

(22) PCT Filed: Feb. 12, 2010
(86) PCT No.: PCT/IB2010/050652

§ 371 Date: Oct. 6, 2011

(30) Foreign Application Priority Data
Feb. 12, 2009 (ZA) 2009/01000

(51) Int. Cl. A61B 19/00

(52) U.S. Cl. 606/204.35; 606/201

(57) ABSTRACT

This invention relates to a device for tightening skin of a human subject to provide a more youthful appearance. The device (10) includes an anchor element (12) that is secured around a part of a human subject. Adhesive elements (18) are provided for releasably adhering to the subject's skin adjacent the area of skin to be tightened. A tensile element (16) extends between the adhesive element (18) and the belt (12). In use, the adhesive elements (18) are adhered to the skin by removing the backing sheet covering the adhesive surface (26), and pressing the adhesive surface (26) to the skin close to the area where the subject wishes to have the skin appear smoother and more youthful. The adhesive elements (18) are pulled towards the anchor element (12) by the tensile elements (16); thereby stretching the skin. The tensile elements (16) are then secured to the anchor element (12).

### FIELD OF THE INVENTION

This invention relates to improvement of the appearance of a person's skin. More particularly, this invention relates to a device for enhancing the appearance of a person.

### BACKGROUND TO THE INVENTION

As the population ages, more people are becoming concerned with maintaining a youthful appearance.

With advancing age, human skin becomes less elastic and tends to form unsightly folds and wrinkles and/or to lose its tone and thus its smooth appearance. Various solutions to achieve a more youthful appearance are offered by the beauty industry, such as topical ointments, laser therapy, oral supplements and cosmetic surgery.

Cosmetic surgery is a subspecialty of surgery that uniquely restricts itself to the enhancement of appearance through surgical and medical techniques. It is specifically concerned with maintaining normal appearance, restoring it, or enhancing it beyond the average level toward some aesthetic ideal. Various cosmetic surgical procedures require removal of excess tissue from the human body and pulling the remaining tissue back to a youthful tautness. Some popular prevalent aesthetic or cosmetic procedures are face lifts, abdominal etching, breast lifts and chemical peels. All of these are known to reduce the appearance of wrinkles on the body.

Although many people are anxious to obtain the benefits of cosmetic surgery, they wish to avoid the expense, pain, loss of time and potential for unpleasant side effects associated with such surgical procedures.

The present invention seeks to provide a less invasive and more affordable method to achieve a more youthful and pleasing appearance of a person's skin.

### SUMMARY OF THE INVENTION

According to the present invention there is provided a device for tightening an area of skin of a human subject to provide a firmer and more youthful appearance without the need for surgery, said device including
an anchor element that is releasably attachable to a predetermined part of the human subject's body, to hold the device in place relative to said part of the subject's body,
an adhesive element that includes means for adhering releasably to the subject's skin adjacent said area of skin to be tightened, and
a tensile element that extends between the anchor element and the adhesive element, characterised in that said tensile element can be placed under tension to draw the adhesive element from a first position wherein it is adhered to subject's skin, towards the anchor element, to a second position, said movement of the adhesive element under tension from the first position to the second position stretching the subject's skin in said area of skin to be tightened.

Said anchor element may encircle said part of the subject's body.

Said anchor element may include an elongate part that can encircle said part of the subject's body and said anchor element may include length adjustment means, configured to adjust the length of said elongate part.

Said tensile element may include length adjustment means.

Said tensile element may be removably attachable to the adhesive element, and said tensile element may include releasable attachment means for attaching it to the adhesive element.

Said tensile element may be removably attachable to the anchor element, and said tensile element may include releasable attachment means for attaching it to the anchor element

Said tensile element may be made of an elastic material.

Said anchor element may be made of an elastic material.

Said adhesive element may be made of an elastic material, and may have an adhesive coated surface for adhering to the subject's skin.

Said anchor element may be an item of clothing.

Said anchor element may include attachment means for attaching it to an item of clothing.

Said anchor element may be a male hook-and-loop fastening strip.

Said tensile element may be a female hook-and-loop fastening strip, and said tensile element may be removably attachable to an outer surface of said adhesive element.

Said tensile element may be removably attachable to said anchor element.

Said anchor element may be a hair accessory for securing the device in said subject's hair, and said tensile element may comprise hair that is removably attachable to an outer surface of said adhesive element.

Said item of clothing may be underwear, and said underwear may be a brassiere.

Said item of clothing may be a shirt, pants or a skirt.

### BRIEF DESCRIPTION OF THE DRAWINGS

For a better understanding of the present invention, and to show how the same may be carried into effect, the invention will now be described by way of a non-limiting example, with reference to the accompanying drawings, in which:

FIG. 1 is a three dimensional view of a skin tightening device in accordance with a first embodiment of the present invention;

FIG. 2 is a three dimensional view of a skin tightening device in accordance with a second embodiment of the invention; and

FIG. 3 is a schematic illustration of a skin tightening device in accordance with a third embodiment of the invention.

### DETAILED DESCRIPTION OF THE DRAWINGS

Referring to the drawings, reference numeral 10 generally indicates a device for tightening an area of skin of a human subject to provide a firmer and more youthful appearance without the need for surgery according to a first embodiment of the invention.

The skin tightening device 10 includes an anchor element that can be in the form of a belt 12 which is made of a flexible fabric or elastic material that can be secured around a part of a human body (not shown). The length of the belt 12 can be adjusted to fit comfortably around the body of a person by length adjustment means in the form of an adjustable clasp 14. The clasp 14 can also be replaced by a hook formation, a hook-and-loop fastener (e.g. Velcro™), or any other suitable adjustment means. Alternatively, the .anchor element 12 can be adapted to be removably securable to another garment such as a shirt or a brassiere (not shown).

Adhesive elements 18 are provided for releasably adhering to the subject's skin adjacent the area of skin to be tightened. Each adhesive element 18 is removably attached to a tensile element 16 with releasable attachment means 20. The tensile element extends between the adhesive element 18 and the belt 12, and is removably attached to the belt 12 with releasable attachment means 22.

The attachment means 20 22 may be hook-and-loop fasteners (e.g. Velcro™), clasps, belts, hook formations, or safety pins. The tensile element 16 can be made of a non flexible fabric, a flexible fabric or an elastic material, and includes length adjustment means in the form of an adjustable buckle 24 for adjusting the tension thereof. Other suitable length adjustment means can also be used.

The adhesive elements 18 can be made of a transparent or flesh-coloured flexible fabric and/or elastic material that include an adhesive coated surface 26 for adhering to the skin to be stretched. The adhesive coated surface 26 have a protective backing sheet. The adhesive coated surface 26 have sufficient area to maintain adherence to the skin with the tensile elements 16 under tension. The belt 12 and the adhesive elements 18 can have lace trimmings, thereby creating the appearance of a garter belt.

In use, the belt 12 is tied and secured around the body of a human subject near the area of skin that the subject wishes to have a firmer and more youthful appearance. The belt 12 can be tied around the waist, upper body, upper arm, upper leg or any other body part of the subject, or can be secured to another garment such as a brassiere or a shirt. The adhesive elements 18 are then attached to the tensile elements 16 by means of the attachment means 20. Thereafter each adhesive element 18 is adhered to the skin by removing the backing sheet covering the adhesive surface 26, and pressing the adhesive surface 26 to the skin close to the area where the subject wishes to have the skin appear smoother and more youthful.

Said tensile elements 16 are then placed under tension by pulling them towards the belt 12, thereby pulling the adhesive elements 18 from a first position to a second position, and the movement of the adhesive element 18 under tension from the first position to the second position stretching the subject's skin in said area of skin to be tightened.

The tensile elements 16 are then secured to the belt 12 through the attachment means 22. The tension in the tensile elements 16 can be adjusted at the adjustable buckles 24 until a desired stretching effect is achieved to give the skin a more youthful and firmer appearance. The belt 12 can be hidden by an item of clothing (not shown).

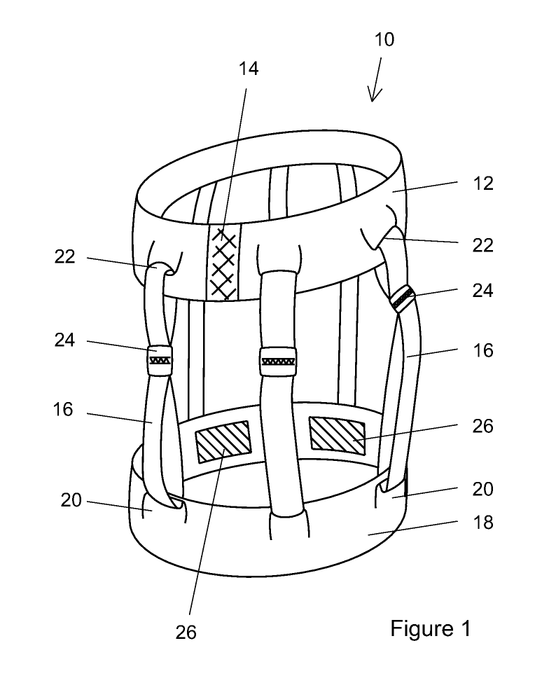

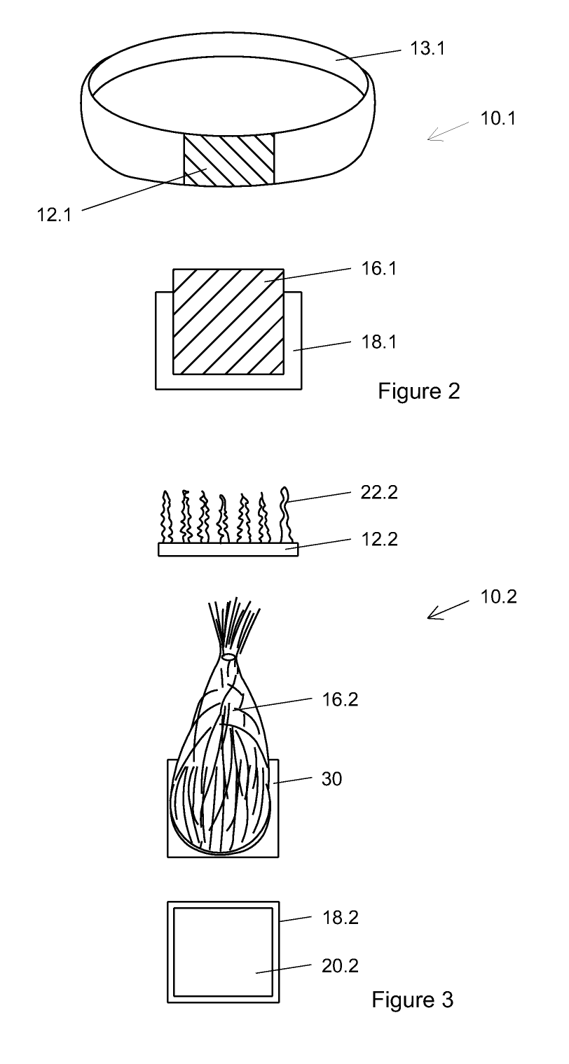

Referring to FIG. 2 of the drawings, reference numeral 10.1 generally shows a skin tightening device in accordance with a second embodiment of the invention. The skin tightening device 10.1 has components in common with the skin tightening device of FIG. 1. Like parts have been designated with like numerals to which the suffix “0.1” has been added.

The skin tightening device 10.1 includes an anchor element in the form of a male hook-and-loop fastener 12.1 that can be secured to a garment 13.1 in such a manner that the garment 13.1 hides the male hook-and-loop fasteners. The garment 13.1 can be a shirt, a belt, a brassiere or an undergarment such as hot pants.

A tensile element 16.1 is provided in the form of a female hook-and-loop fastener 16.1. The female hook-and-loop fastener 16.1 is secured to a top surface of an adhesive element 18.1. The adhesive element 18.1 is provided in the form of a flexible fabric and/or elastic material that includes an adhesive coated surface (not shown) for adhering to the skin to be stretched. The adhesive coated surface (not shown) has a protective backing sheet, and the surface has sufficient area to maintain adherence to the skin when the adhesive element 18.1 is placed under tension.

In use, the adhesive element 18.1 is adhered to the skin by removing the backing sheet covering the adhesive coated surface and pressing the adhesive coated surface to the skin next to the area where the subject wishes the skin to appear smoother and more youthful. The subject then pulls the female hook-and-loop fastener 16.1 towards the male hook-and-loop fastener 12.1, thereby creating tension to stretch loose skin. The female hook-and-loop fastener 16.1 is then secured to the male hook-and-loop fastener 12.1.

The tension in the female hook-and-loop fastener 16.1 can be adjusted by varying the distance between the adhesive element 18.1 and the male hook-and-loop fastener 12.1 until a desired stretching effect is achieved to give the skin a more youthful and firmer appearance.

Referring to FIG. 3 of the drawings, reference numeral 10.2 generally shows a skin tightening device in accordance with a third embodiment of the invention. The skin tightening device 10.2 has components in common with the skin tightening device of FIGS. 1 and 2. Like parts have been designated with like numerals to which the suffix “0.2” has been added.

The skin tightening device 10.2 includes an anchor element in the form of a hairclip 12.2. A tensile element 16.2 is provided in the form of hair that is secured to a male hook-and-loop fastener 30 in such a manner that the hair hides the male hook-and-loop fastener 30.

An adhesive element 18.2 in the form of a flexible fabric and/or elastic material is provided that includes an adhesive coated surface (not shown) for adhering to the skin to be stretched. The adhesive coated surface (not shown) has a protective backing sheet, and the surface has sufficient area to maintain adherence to the skin when the adhesive element 18.2 is placed under tension. A female hook-and-loop fastener 20.2 is attached to a top surface of the adhesive element 18.2.

In use, the adhesive element 18.2 is adhered to the skin by removing the backing sheet covering the adhesive coated surface and pressing the adhesive coated surface to the skin above the area where the subject wishes to have the skin appear smoother and more youthful. The subject then secures the male hook-and-loop fastener 30 to the female hook-and-loop fastener 20.2 and pulls the hair 16.2, resulting in the skin being stretched. The hair 16.2 can be braided with the subjects own hair before it is secured with the teeth 22.2 of the hairclip 12.2. Alternatively, the hair 16.2 can be glued to the subjects own hair.

The amount that the skin that is stretched can be adjusted by varying the distance between the adhesive element 18.2 and the hairclip 12.2 until a desired stretching effect is achieved to give the skin a more youthful and firmer appearance.

1. A device (10) for tightening an area of skin of a human subject to provide a firmer and more youthful appearance without the need for surgery, said device (10) including:
an anchor element (12) that is releasably attachable to a predetermined part of the human subject's body, to hold the device (10) in place relative to said part of the subject's body;
an adhesive element (18) that includes means for adhering releasably to the subject's skin adjacent said area of skin to be tightened, and
a tensile element (16) that extends between the anchor element (12) and the adhesive element (18);
characterised in that said tensile element (16) can be placed under tension to draw the adhesive element (18) from a first position wherein it is adhered to subject's skin, towards the anchor element (12), to a second position, said movement of the adhesive element (18) under tension from the first position to the second position stretching the subject's skin in said area of skin to be tightened.

2. A device as claimed in claim 1, wherein said anchor element (12) can encircle said part of the subject's body.

3. A device as claimed in claim 2, wherein said anchor element (12) includes an elongate part that can encircle said part of the subject's body and said anchor element (12) has length adjustment means (14), configured to adjust the length of said elongate part.

4. A device as claimed in any one of claim 1 to 3, wherein said tensile element (16) includes length adjustment means (24).

5. A device as claimed in any one of the preceding claims, wherein said tensile element (16) is removably attachable to the adhesive element (18).

6. A device as claimed in claim 5, wherein said tensile element (16) includes releasable attachment means (20) for attaching it to the adhesive element (18).

7. A device as claimed in any one of the preceding claims, wherein said tensile element (16) is removably attachable to the anchor element (12).

8. A device as claimed in claim 7, wherein said tensile element (16) includes releasable attachment means (22) for attaching it to the anchor element (12).

9. A device as claimed in any one of the preceding claims, wherein said tensile element (16) is made of an elastic material.

10. A device as claimed in any one of the preceding claims, wherein said anchor element (12) is made of an elastic material.

11. A device as claimed in any one of the preceding claims, wherein said adhesive element (18) is made of an elastic material.

12. A device as claimed in any one of the preceding claims, wherein said adhesive element (18) has an adhesive coated surface (26) for adhering to the subject's skin.

13. A device as claimed in claim 1, wherein said anchor element (12) is an item of clothing.

14. A device as claimed in any one of claim 1 to 12, wherein said anchor element (12) includes attachment means for attaching it to an item of clothing.

15. A device as claimed in claim 1, wherein said anchor element (12.1) is a male hook-and-loop fastening strip.

16. A device as claimed in claim 15, wherein said tensile element (16.1) is a female hook-and-loop fastening strip.

17. A device as claimed in claim 16, wherein said tensile element (16.1) is removably attachable to an outer surface of said adhesive element (18.1).

18. A device as claimed in claim 16 or claim 17, wherein said tensile element (16.1) is removably attachable to said anchor element (12.1).

19. A device as claimed in claim 1, wherein said anchor element (12.2) is a hair accessory for securing the device (10.2) in said subject's hair.

20. A device as claimed in claim 19, wherein said tensile element (16.2) comprises hair that is removably attachable to an outer surface of said adhesive element (18.2).

21. A device as claimed in claim 13 or claim 14, wherein said item of clothing is underwear.

22. A device as claimed in claim 21, wherein said underwear is a brassiere.

23. A device as claimed in claim 13 or claim 14, wherein said item of clothing is a shirt.

24. A device as claimed in claim 13 or claim 14, wherein said item of clothing is a skirt

25. A device as claimed in claim 13 or claim 14, wherein said item of clothing is pants.

26. A device as claimed in any one of the preceding claims, substantially as herein described with reference to and as illustrated in any one of the drawings.
